(19) United States
(12) Patent Application Publication
(10) Pub. No.: US 2026/0118333 A1
(43) Pub. Date: Apr. 30, 2026

(54) HYBRID HYDROGEN SENSOR AND METHOD OF CONTROLLING THE SAME

(71) Applicants: Hyundai Motor Company, Seoul (KR); Kia Corporation, Seoul (KR)

(72) Inventor: Jin Kyo JEONG, Hwaseong-Si (KR)

(21) Appl. No.: 19/198,547

(22) Filed: May 5, 2025

(30) Foreign Application Priority Data
Oct. 28, 2024 (KR) 10-2024-0148910

(51) Int. Cl. G01N 33/00; G01D 21/02; G01N 25/18; G01N 25/56; G01N 27/16; G01N 27/18

(52) U.S. Cl. CPC G01N 33/005; G01D 21/02; G01N 25/18; G01N 25/56; G01N 27/18; G01N 27/16

(57) ABSTRACT

The present disclosure relates to a hybrid hydrogen sensor and a method of controlling the same. The method may include applying power to a hybrid hydrogen sensor comprising a resistive hydrogen sensor and a thermal conduction hydrogen sensor; obtaining, from the resistive hydrogen sensor, a first output value; obtaining, from the thermal conduction hydrogen sensor, a second output value; comparing the first output value with a predetermined saturation output value; estimating at least one of a concentration of hydrogen or humidity based on one or more selections, according to a result of the comparison, from the first output value and the second output value; and outputting, based on the estimating, a signal indicating the at least one of the concentration of hydrogen or the humidity for a fuel cell.

### CROSS-REFERENCE TO RELATED APPLICATION

This application claims priority to Korean Patent Application No. 10-2024-0148910, filed in the Korean Intellectual Property Office on Oct. 28, 2024, the entire contents of which are incorporated herein by reference.

### TECHNICAL FIELD

The present disclosure relates to hydrogen sensors.

### BACKGROUND

Carbon neutrality is a topic of interest for many industries and policy makers around the world.

Major economies are seeking ways for expanding electricity production using renewable energy instead of traditional fossil fuel.

An energy system that harnesses various forms of renewable energy such as wind power, hydro power, tidal power, and solar power and convert them into electrical or hydrogen energy is sometimes called a green energy system.

Among them, the so-called “green hydrogen” is considered by many to be the ultimate eco-friendly energy because it emits no greenhouse gas at a production stage. Hydrogen, which is emerging as an alternative energy of choice on a global scale, can be further classified into gray hydrogen, blue hydrogen, and green hydrogen depending on a production method.

As electric vehicles become more widely adopted, research on electric vehicles equipped with eco-friendly hydrogen fuel cells has been actively conducted.

Hydrogen fuel-based electric vehicles may provide a fast charging time and a long driving distance with a single hydrogen charge due to high energy density of hydrogen fuel as compared to electric vehicles equipped with high-voltage batteries.

A fuel cell system generates electricity through a chemical reaction between hydrogen and oxygen in a stack. However, the hydrogen has a very wide explosive range (e.g., flammability limits) of 4% to 75% and can be ignited only with 0.20 mJ of energy in the atmosphere, and thus, the risk of explosion in using a fuel cell can be a an issue. Further, the hydrogen has a very high diffusion coefficient (0.61 cm²/s in the air) and is easily diffused through most materials, making it very difficult to trap the hydrogen in a specific container. As a result, devices or accessories related to hydrogen require special attention to hydrogen leakage. Thus, a hydrogen leakage sensor capable of detecting a hydrogen concentration of approximately 1000 ppm to 4% should be equipped on a vehicle in order to utilize fuel cell. Further, a hydrogen sensor capable of identifying a concentration of the hydrogen supplied to a fuel cell stack may also be required. The concentration of the hydrogen may vary between 40% and 99.9% according to a design of the fuel cell system. Thus, there is a need for development of the hydrogen sensor capable of efficiently detecting the concentration of the hydrogen.

However, in some implementations of hydrogen-related devices and accessories, a resistive hydrogen sensor, capable of measuring a low hydrogen concentration, or a thermal conduction hydrogen sensor, capable of measuring a high hydrogen concentration, is selectively applied. Thus, when the concentration of the hydrogen deviates from a detection range of the corresponding hydrogen sensor, the system may malfunction or cause a serious accident.

### SUMMARY

The present disclosure has been made to solve the above-mentioned problems occurring in at least some implementations while advantages achieved by those implementations are maintained intact.

An aspect of the present disclosure provides a hybrid hydrogen sensor having measurement accuracy and a wide detection range for a concentration of hydrogen, and a method of controlling the same.

Another aspect of the present disclosure provides a hybrid hydrogen sensor capable of measuring a wide range of a concentration of hydrogen in a hybrid detecting manner by coupling a resistive hydrogen sensor, based on a hydrogen-detecting material, and a thermal conduction hydrogen sensor, based on a micro-electromechanical systems (MEMS) heater, into a single element, and a method of controlling the same.

Still another aspect of the present disclosure provides a hydrogen sensor having a wide detecting range with high accuracy without an additional compensation element by applying a hybrid detecting method on a single element.

Yet another aspect of the present disclosure provides a hybrid hydrogen sensor capable of determining whether a sensor is normal based on comparison between output values of two sensing logics on a single element, and a method of controlling the same.

The technical problems to be solved by the present disclosure are not limited to the aforementioned problems, and any other technical problems not mentioned herein will be clearly understood from the following description by those skilled in the art to which the present disclosure pertains.

According to one or more example embodiments of the present disclosure, a method may include: applying power to a hybrid hydrogen sensor including a resistive hydrogen sensor and a thermal conduction hydrogen sensor; obtaining, from the resistive hydrogen sensor, a first output value; obtaining, from the thermal conduction hydrogen sensor, a second output value; comparing the first output value with a predetermined saturation output value; estimating at least one of a concentration of hydrogen or humidity based on one or more selections, according to a result of the comparison, from the first output value and the second output value; and outputting, based on the estimating, a signal indicating the at least one of the concentration of hydrogen or the humidity for a fuel cell.

The resistive hydrogen sensor may be based on a hydrogen detecting material. The thermal conduction hydrogen sensor may be based on a micro heater platform. The resistive hydrogen sensor and the thermal conduction hydrogen sensor may be implemented as a single element.

Estimating the at least one of the concentration of hydrogen or the humidity may include: estimating, based on the result of the comparison indicating that the first output value is greater than the predetermined saturation output value, the concentration of hydrogen based on the second output value.

Estimating the at least one of the concentration of hydrogen or the humidity may include: based on the result of the comparison indicating that the first output value is less than or equal to the predetermined saturation output value: estimating the humidity based on the first output value and the second output value; and estimating the concentration of hydrogen based on the estimated humidity.

The hybrid hydrogen sensor may include a micro heater platform. The method may further include determining an environmental temperature based on a resistance value of a temperature sensor array formed in the micro heater platform. The humidity may be estimated further based on the environmental temperature.

The method may further include: performing constant temperature control by applying a current to a heater array formed in the micro heater platform after the determining of the environmental temperature. The first output value and the second output value may be obtained based on the performing of the constant temperature control.

The method may further include one of: outputting, based on a low concentration state in which the first output value is less than or equal to the predetermined saturation output value, an environmental temperature, the humidity, and the concentration of hydrogen; or outputting, based on a high concentration state in which the first output value is greater than the predetermined saturation output value, the environmental temperature and the concentration of hydrogen.

The method may further include: setting a timer after the outputting; and determining, based on the timer being expired, a second environmental temperature.

The hybrid hydrogen sensor may include a hydrogen detecting material and a micro heater platform. The method may further include: estimating, based on a determination that the micro heater platform does not include a temperature sensor array, an environmental temperature by measuring a resistance value of a heater array formed at the micro heater platform in a heater OFF state.

The resistive hydrogen sensor may include a hydrogen detecting material. The predetermined saturation output value may be determined based on hydrogen reaction characteristics of the hydrogen detecting material.

According to one or more example embodiments of the present disclosure, a hybrid hydrogen sensor may include: an insulating layer in which a micro heater platform is formed; a substrate disposed on a first surface of the insulating layer; a hydrogen detecting material layer disposed on a second surface of the insulating layer; a processor disposed on the substrate; and a memory storing at least one instruction. The at least one instruction, when executed by the processor communicating with the memory, may be configured to cause the hybrid hydrogen sensor: compare a first sensor output value that is based on the hydrogen detecting material layer with a predetermined saturation output value; estimate at least one of a concentration of hydrogen or humidity based on one or more selections, according to a result of the comparison, from the first sensor output value and a second sensor output value that is based on the micro heater platform; and output, based on the estimation, a signal indicating the at least one of the concentration of hydrogen or the humidity for a fuel cell.

The micro heater platform may include a heater array and a temperature sensor array formed on a same plane.

The at least one instruction, when executed by the processor communicating with the memory, may be configured to cause the hybrid hydrogen sensor to estimate the at least one of the concentration of hydrogen or the humidity by: estimating, based on the result of the comparison indicating that the first sensor output value is greater than the predetermined saturation output value, the concentration of hydrogen based on the second sensor output value.

The at least one instruction, when executed by the processor communicating with the memory, may be configured to cause the hybrid hydrogen sensor to estimate the at least one of the concentration of hydrogen or the humidity by: based on the result of the comparison indicating that the first sensor output value is less than or equal to the predetermined saturation output value: estimating the humidity based on the first sensor output value and the second sensor output value; and estimating the concentration of hydrogen based on the estimated humidity.

The at least one instruction, when executed by the processor communicating with the memory, may be configured to further cause the hybrid hydrogen sensor to: determining an environmental temperature based on a resistance value of the temperature sensor array. Estimating the humidity may be further based on the environmental temperature.

The at least one instruction, when executed by the processor communicating with the memory, may be configured to further cause the hybrid hydrogen sensor to: perform constant temperature control by applying a current to the heater array after the determining of the environmental temperature; and obtain the first sensor output value and the second sensor output value based on the performing of the constant temperature control.

The at least one instruction, when executed by the processor communicating with the memory, may be configured to further cause the hybrid hydrogen sensor to perform one of: outputting, based on a high concentration state in which the first sensor output value is greater than the predetermined saturation output value, an environmental temperature and the concentration of hydrogen; or outputting, based on a low concentration state in which the first sensor output value is less than or equal to the predetermined saturation output value, the environmental temperature, the concentration of hydrogen, and the humidity.

The at least one instruction, when executed by the processor communicating with the memory, may be configured to further cause the hybrid hydrogen sensor to: set a timer after the outputting; and determine, based on the timer being expired, a second environmental temperature.

The at least one instruction, when executed by the processor communicating with the memory, may be configured to further cause the hybrid hydrogen sensor to: estimate, based on a determination that the micro heater platform does not include a temperature sensor array, an environmental temperature by measuring a resistance value of a heater array formed at the micro heater platform in a heater OFF state.

The hydrogen detecting material layer may include a hydrogen detecting material. The predetermined saturation output value may be determined based on hydrogen reaction characteristics of the hydrogen detecting material. The hydrogen detecting material may include palladium.

According to one or more example embodiments of the present disclosure, a hybrid hydrogen sensor may include: a micro heater platform; a hydrogen detecting material layer; a processor; and a memory storing at least one instruction. The at least one instruction, when executed by the processor communicating with the memory, may be configured to cause the hybrid hydrogen sensor to: compare a first sensor output value that is based on the hydrogen detecting material layer with a predetermined saturation output value; estimate at least one of a concentration of hydrogen or humidity based on one or more selections, according to a result of the comparison, from the first sensor output value and a second sensor output value that is based on the micro heater platform; and output, based on the estimation, indicating the at least one of the concentration of hydrogen or the humidity for a fuel cell.

### BRIEF DESCRIPTION OF THE DRAWINGS

The above and other objects, features and advantages of the present disclosure will be more apparent from the following detailed description taken in conjunction with the accompanying drawings:

FIG. 1 is a view for describing an example electrochemical reaction in a fuel cell stack;

FIG. 2 shows graphs for characteristics of example hydrogen sensing methods applied to a hybrid hydrogen sensor;

FIG. 3 is a view for describing an example configuration of the hybrid hydrogen sensor;

FIG. 4 is a view for describing an example structure of the hybrid hydrogen sensor;

FIG. 5 is a flowchart showing an example method of controlling the hybrid hydrogen sensor;

FIG. 6 is a view for describing an example configuration of a hybrid hydrogen sensor;

FIG. 7 is a view for describing an example structure of the hybrid hydrogen sensor;

FIG. 8 is a flowchart showing an example method of controlling the hybrid hydrogen sensor;

FIG. 9 is an example output of a resistive hydrogen sensor and a thermal conduction hydrogen sensor;

FIG. 10 is an example output of the hybrid hydrogen sensor; and

FIG. 11 is a structure of an example computing device.

### DETAILED DESCRIPTION

One or more example embodiments of the present disclosure will be described in detail with reference to the exemplary drawings. In adding reference numerals to components of each drawing, it should be noted that identical or equivalent components are designated by an identical numeral even when they are displayed on other drawings. Further, in describing the example embodiments of the present disclosure, a detailed description of the related known configuration or function will be omitted when it is determined that it interferes with the understanding of the example embodiments of the present disclosure.

In describing the components of the example embodiments according to the present disclosure, terms such as first, second, A, B, (a), (b), and the like may be used. These terms are merely intended to distinguish one component from other components, and the terms do not limit the nature, order, or sequence of the components. Unless otherwise defined, all terms including technical and scientific terms used herein include the same meaning as commonly understood by one of ordinary skill in the art to which the present disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

For purposes of this application and the claims, using the exemplary phrase “at least one of: A; B; or C” or “at least one of A, B, or C,” the phrase means “at least one A, or at least one B, or at least one C, or any combination of at least one A, at least one B, and at least one C. Further, exemplary phrases, such as “A, B, or C”, “at least one of A, B, and C”, “at least one of A, B, or C”, etc. as used herein may mean each listed item or all possible combinations of the listed items. For example, “at least one of A or B” may refer to (1) at least one A; (2) at least one B; or (3) at least one A and at least one B.

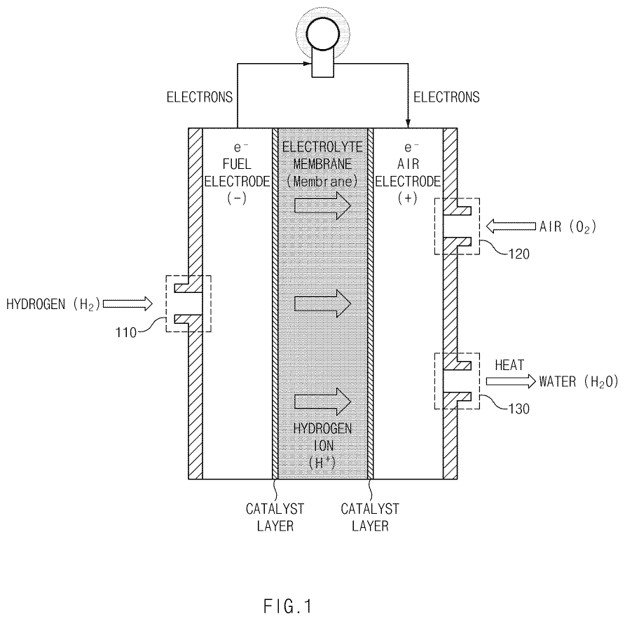

Hereinafter, one or more example embodiments of the present disclosure will be described in detail with reference to FIGS. 1 to 11.

FIG. 1 is a view for describing an example electrochemical reaction in a fuel cell stack.

Referring to FIG. 1, a fuel cell stack 100 may include a fuel electrode as a negative electrode (e.g., anode), an air electrode as a positive electrode (e.g., cathode), and an electrolyte membrane disposed between the fuel electrode and the air electrode.

Hydrogen injected into the fuel electrode of the fuel cell stack 100 and oxygen injected into the air electrode may react electrochemically so as to constantly generate water (e.g., electrolyzed water, H₂O).

Catalyst layers may be formed on either side (e.g., front and back) of the electrolyte membrane to allow a chemical reaction to occur in the fuel cell stack. For example, the catalyst layer may be formed using carbon powder coated with a platinum (Pt)-based catalyst, but this is merely an example, and other catalyst materials may be used depending on a design of those skilled in the art. The catalyst layer may form a gas diffusion layer using a catalyst.

The hydrogen and the oxygen injected to a left side (e.g., the fuel electrode side) and a right side (e.g., the air electrode side) of the stack may be ionized through oxidation and reduction processes.

When hydrogen gas (H₂) is injected through a hydrogen inlet 110 formed at one location on the fuel electrode, the hydrogen may react with the catalyst and be decomposed into hydrogen ions (H⁺) and electrons (e⁻). This reaction may be represented by the chemical reaction formula, 2H₂→4H⁺+4e⁻.

The hydrogen ions may pass through the electrolyte membrane and move to the air electrode. The electrons generated from the fuel electrode may pass through an external circuit and generate a current. In this case, a motor of an electric vehicle may be driven (e.g., powered) using the generated current. A current generated in the fuel cell stack may be used to charge a battery provided in the electric vehicle.

When oxygen gas (O₂) is injected through an air inlet 120 formed at one location on the air electrode, the oxygen (O₂) and electrons (4e⁻) may react with the help of the catalyst to generate oxygen ions (2O²⁻). The generated oxygen ions (2O²⁻) and hydrogen ions (4H⁻) passing through the electrolyte membrane may react to generate water (2H₂O). The water and heat generated during a chemical reaction in the cell may be discharged out of the fuel cell stack through a water outlet 130 formed at another location on the air electrode.

A hybrid hydrogen sensor may be disposed in the hydrogen inlet 110 of the fuel cell stack 100 to measure a concentration of hydrogen supplied to the fuel cell stack 100, but this is merely an example, and depending on a design of those skilled in the art, the hybrid hydrogen sensor may be disposed at another location in a fuel cell system, in a fuel cell vehicle system, in a vehicle, around a stack or a hydrogen tank, at any location where there is a risk of hydrogen leakage. The hybrid hydrogen sensor may be used as a hydrogen leakage detection sensor.

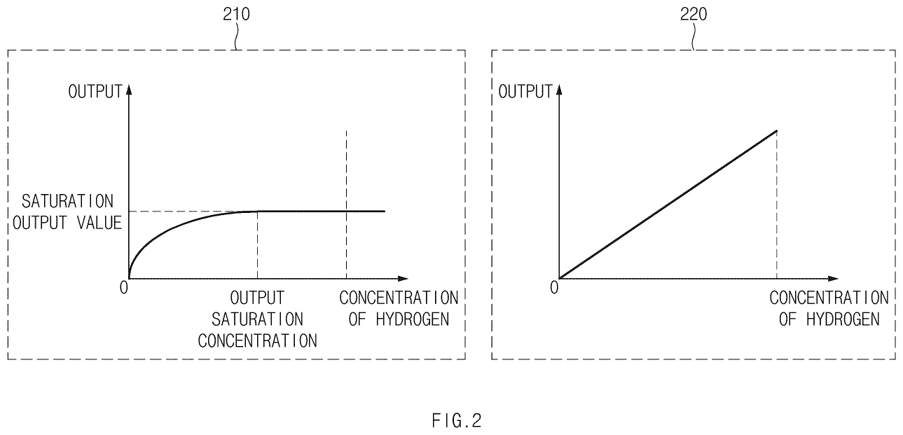

FIG. 2 shows graphs for characteristics of example hydrogen sensing methods applied to a hybrid hydrogen sensor.

The hybrid hydrogen sensor may be implemented by coupling a hydrogen detecting material-based resistive hydrogen sensor and a MEMS heater-based thermal conduction hydrogen sensor into a single element.

The resistive hydrogen sensor (also referred to as a resistive sensor or a resistance change-type hydrogen sensor) may be a hydrogen sensor that detects the presence or absence (or a concentration) of the hydrogen in the surrounding atmosphere based on the amount of change in a material resistance using a detection material that has excellent hydrogen selective reactivity. Generally, the resistive hydrogen sensor may not exhibit a large resistance change between dry air and wet air, but exhibit fine reactivity degradation in the wet air due to obstruction of water vapor in the air when the same hydrogen concentration reaction is performed on the dry air and the wet air. Therefore, securing of reaction performance (e.g., a reaction speed) optimization of a driving condition for removing a humidity effect (e.g., an optimum driving temperature) or improvement of a material (e.g., surface hydrophobicity treatment and nano-structure optimization) may be required. A heater composite structure for heating to an optimum temperature may be applied.

The resistive hydrogen sensor may measure the amount of change saturated in an environment having a specific hydrogen concentration or more according to chemical reaction saturation characteristics of the detection material.

Palladium (Pd) may be used as the hydrogen detecting material, but the present disclosure is not limited thereto.

When palladium is exposed to a hydrogen gas, the hydrogen gas may be dissociated from a surface of palladium and be absorbed into palladium. Palladium may dissolve the hydrogen up to 600 times in a volume ratio, and the absorbed hydrogen reacts with palladium to form a hydrogen compound of PdHx. Here, x may be determined by a partial pressure of the hydrogen, and accordingly, mechanical, optical, and/or electrical characteristics of PdHx are changed according to the partial pressure of the hydrogen. Thus, the concentration of the hydrogen may be measured by measuring changes in the electrical, mechanical, and/or optical characteristics of PdHx. Further, palladium may measure only the concentration of the hydrogen without being affected by other gases and may be used in a vacuum state or an environment in which there is no oxygen.

In the case of pure palladium, a resistance change amount may be saturated at a hydrogen concentration of about 2% to 4% or more due to a limit of phase transition of the PdHx. That is, the hydrogen concentration may not be measured at a high concentration that is greater than or equal to the hydrogen concentration at which the resistance change amount starts to be saturated. To overcome these characteristics, alloying or structural improvement (e.g., laminated thin films, nano-structures, or the like) may be suggested as a way to secure detection linearity at a high concentration greater than or equal to 4% may be achieved.

Reference numeral 210 indicates output according to a hydrogen concentration of the hydrogen detecting material-based resistive hydrogen sensor. As indicated by reference numeral 210, it may be identified that, when the hydrogen concentration is greater than or equal to an output saturation concentration, the output is saturated and the output is no longer increased even when the hydrogen concentration is increased.

The MEMS heater-based thermal conduction hydrogen sensor (also referred to as a thermal conduction sensor or a conduction-type hydrogen sensor) may be a hydrogen sensor that detects the presence or absence (or the concentration) of the hydrogen in the surrounding atmosphere by measuring a change in thermal conductivity characteristics to the surrounding atmosphere when the MEMS heater is driven (e.g., powered) using high thermal conductivity characteristics of the hydrogen as compared to other gases. As an example, the presence or absence (or the concentration) of the hydrogen in the atmosphere may be detected by detecting the amount of change in a heater temperature after the heater is driven with power (or specific power) required to reach a specific temperature. In this method, there may be thermal conductivity influences by other gases as well as the hydrogen. In particular, as the concentration of the hydrogen is decreased, a change in the thermal conductivity characteristics due to the hydrogen may not be large, and thus influence on other gases may be large. Here, the influence on other gases may be typically humidity of several percent or more in a general environment, and in this case, compensation may be required using a separate humidity compensation element.

Further, unlike the hydrogen detecting material-based resistive hydrogen sensor, the thermal conduction hydrogen sensor may be based on physical quantities according to the hydrogen concentration in a surrounding environment and thus has linear detecting characteristics in the entire concentration range (e.g., 0% to 100%) as indicated by reference numeral 220.

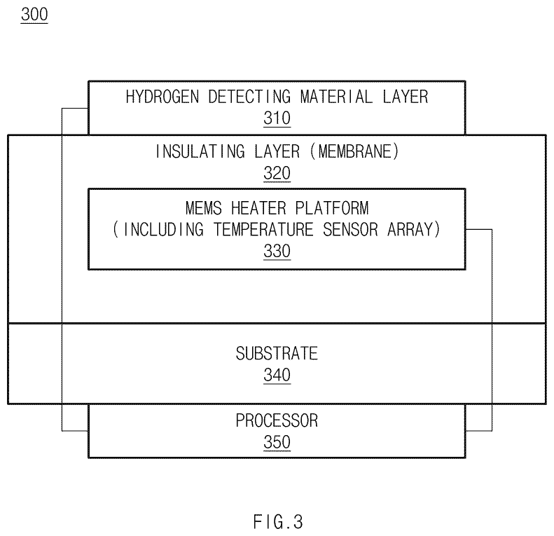

FIG. 3 is a view for describing an example configuration of the hybrid hydrogen sensor.

Referring to FIG. 3, a hybrid hydrogen sensor 300 may include a hydrogen detecting material layer 310, an insulating layer (e.g., membrane) 320, a substrate 340, and a processor 350.

A MEMS heater platform 330 may be formed inside the insulating layer 320. The MEMS heater platform 330 may include one or more temperature sensors (e.g., an array of sensors).

The MEMS heater platform 330 may be disposed inside (or between) the insulating layer 320, thereby securing heating performance of the MEMS heater.

As an example, the insulating layer 320 may be formed of a silicon oxide (SiO₂) layer or a silicon nitride (Si₃N₄) layer.

The MEMS heater platform 330 may be formed such that a heater array (also referred to as a heater pattern) and a temperature sensor array (also referred to as a temperature sensor pattern) are arranged on the same plane. The heater array may include a plurality of heaters. The temperature sensor may include a plurality of sensors. In this case, the heater array and the temperature sensor array may be arranged in a pattern to have a predetermined interval so that a temperature of the surrounding environment may be more accurately measured without being affected by an increase in a temperature caused by a heating operation of the MEMS heater as much as possible.

The heater array and the temperature sensor array may be implemented on the same plane through a patterning technique such as photolithography.

The heater array and the temperature sensor array may be implemented in various micro-patterns such as a meander pattern to secure excellent temperature detecting performance and excellent heating performance.

A relational expression related to a temperature influence on the surrounding environment due to the heating of the MEMS heater may be derived through a previous test, and the processor 350 may correct (e.g., adjust or calibrate) an environmental temperature using the corresponding relational expression. Therefore, the processor 350 may accurately measure the environmental temperature regardless of the heating of the MEMS heater.

The MEMS heater array and the temperature sensor array may be made of the same metal material. As an example, the metal material used in the heater array and the temperature sensor array may be pure platinum (Pt) having high linearity temperature coefficient of resistance (TCR) and chemical stability and capable of Joule heating.

The insulating layer 320 may be disposed on one surface of the substrate 340, and the processor 350 may be disposed on the other surface thereof, but this is merely an example, and the insulating layer 320 and the processor 350 may be arranged not to overlap each other on the same surface of the substrate 340.

The hydrogen detecting material layer 310 may be implemented with pure palladium or a palladium alloy (e.g., Pd—Ni, Pd—Ag, etc.) in the form of a thin film.

The hydrogen detecting material layer 310 and the MEMS heater platform 330 may be electrically connected to the processor 350 through the substrate 340.

The processor 350 may acquire the environmental temperature through the temperature sensor formed in the MEMS heater platform 330.

The processor 350 may detect the presence or absence of the hydrogen and the concentration of the hydrogen by monitoring a change in a resistance of the hydrogen detecting material layer 310. That is, the processor 350 may perform a hydrogen detecting material-based resistive hydrogen sensing operation to calculate a resistive output value (e.g., an output value of the resistive hydrogen sensor) and estimate the current concentration of the hydrogen based on the calculated resistive output value.

The processor 350 may measure the presence or absence of the hydrogen and the concentration of the hydrogen by monitoring a change in a temperature of the MEMS heater platform 330. That is, the processor 350 may perform a MEMS heater-based thermal conduction hydrogen sensing operation to calculate a thermal conduction output value (e.g., an output value of a thermal conduction hydrogen sensor) and estimate the current concentration of the hydrogen based on the calculated thermal conduction output value.

The processor 350 may determine whether the current concentration of the hydrogen is in an output saturation state based on chemical reaction saturation characteristics of a hydrogen detecting material used in the hydrogen detecting material layer 310.

The processor 350 may determine whether to enter a saturation output state based on comparison between a preset saturation output value and the resistive output value according to the corresponding hydrogen detecting material.

The processor 350 may determine that a current state is a saturation output state when the resistive output value is greater than the preset saturation output value, and determine that the current state is a normal output state when the resistive output value is less than or equal to the preset saturation output value. In the following description, the saturation output state and the normal output state are used interchangeably as a high concentration state and a low concentration state, respectively.

If the current concentration of the hydrogen is in the saturation output state (e.g., if the current concentration of the hydrogen is in the high concentration state), the processor 350 may estimate the current concentration of the hydrogen based on the thermal conduction output value.

If the current concentration of the hydrogen is in the normal output state (e.g., if the current concentration of the hydrogen is in the low concentration state), the processor 350 may correct (e.g., adjust or calibrate) the concentration of the hydrogen and humidity based on the thermal conduction output value and the resistive output value.

As an example, the processor 350 may estimate the concentration of the hydrogen based on the resistive output value in the low concentration state and then calculate an expected thermal conduction output value A1 corresponding to the estimated concentration of the hydrogen by applying the expected thermal conduction output value A1 to a preset linear equation as indicated by reference numeral 220 of FIG. 2. The processor 350 may estimate a humidity value based on a difference value between the expected thermal conduction output value A1 and a currently estimated thermal conduction output value A2. Since an influence on humidity is included in the case of the thermal conduction output value, the processor 350 may correct the A2 based on the estimated humidity value and determine the concentration of the hydrogen corresponding to the corrected A2 as a final concentration of the hydrogen.

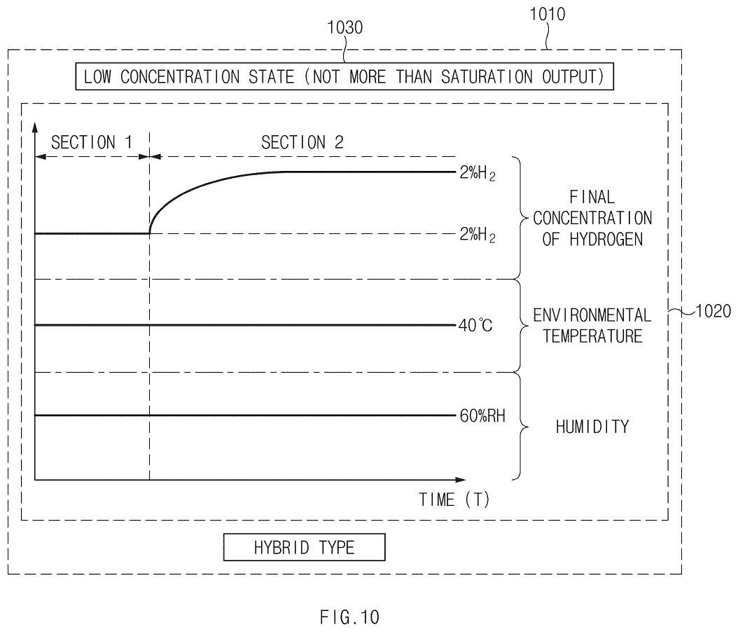

The processor 350 may finally output information on the environmental temperature, the estimated (or corrected) humidity, and the concentration of the hydrogen. The information output by the processor 350 may be output through a screen of measurement equipment connected to the hybrid hydrogen sensor 300, as shown in FIG. 10, which will be described below.

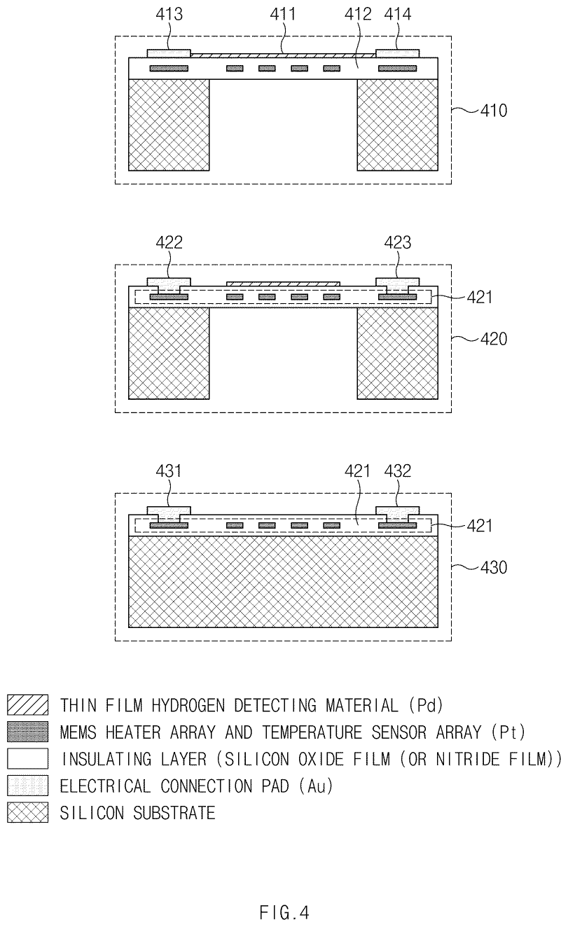

FIG. 4 is a view for describing an example structure of the hybrid hydrogen s.

FIG. 4 shows electrical connection cross sections when the MEMS heater platform of the hybrid hydrogen sensor includes both the heater array and the temperature sensor array.

The heater array and the temperature sensor array may be implemented on the same plane.

Referring to reference numeral 410, a hydrogen detecting material 411 in the form of a thin film may be disposed on one surface of an insulating layer 412, and both ends of the thin film hydrogen detecting material 411 may be connected to first electrical connection pads 413 and 414 formed on the one surface of the insulating layer 412.

Referring to reference numeral 420, a heater array of a MEMS heater platform 421 may be connected to second electrical connection pads 422 and 423 formed on the one surface of the insulating layer 412. The heater array may be formed at (e.g., in or on) the MEMS heater platform 421.

Referring to reference numeral 430, a temperature sensor array of the MEMS heater platform 421 may be connected to third electrical connection pads 431 and 432 formed on the one surface of the insulating layer 412. The temperature sensor array may be formed at (e.g., in or on) the MEMS heater platform 421.

Palladium may be used as the thin film hydrogen detecting material, and platinum may be used as the heater array and the temperature sensor array.

A silicon oxide film or a silicon nitride film may be used as the insulating layer.

The electrical connection pads may be made of gold (Au), but the present disclosure is not limited thereto, and the electrical connection pads may be made of any one of silver (Ag), copper (Cu), aluminum (Al), and an alloy material using them according to a design of the those skilled in the art and the use and purpose of the hybrid hydrogen sensor.

A silicon substrate may be used as the substrate.

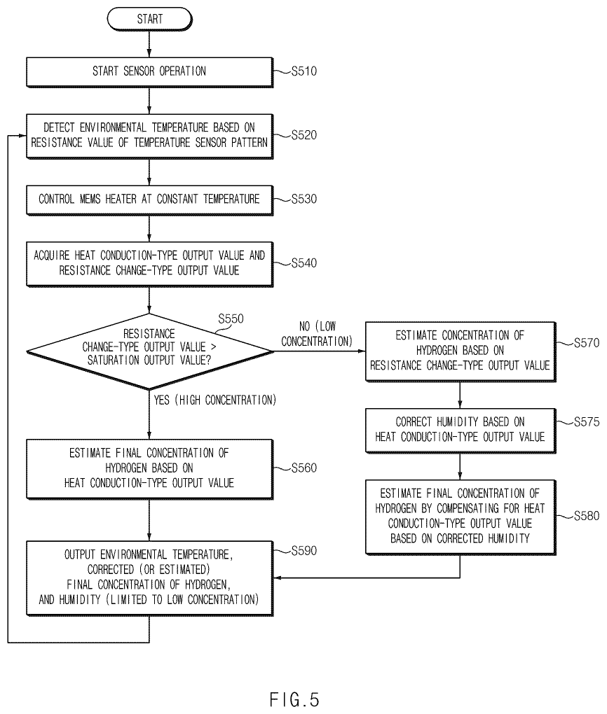

FIG. 5 is a flowchart showing an example method of controlling the hybrid hydrogen sensor.

The controlling method of FIG. 5 may be a method performed by the processor 350 of FIG. 3.

Referring to FIGS. 3 and 5, the processor 350 may start a sensor operation when power is applied to the hybrid hydrogen sensor 300 (S510).

The processor 350 may detect the environmental temperature based on a resistance value of the temperature sensor array of the MEMS heater platform 330 (S520).

The processor 350 may perform constant temperature control for maintaining the MEMS heater at a predefined normal temperature by performing resistance (e.g., temperature) feedback control after power is supplied to the heater array of the MEMS heater platform 330 (S530).

The processor 350 may acquire a MEMS heater-based thermal conduction output value and a hydrogen detecting material-based resistive output value (S540).

The processor 350 may compare the resistive output value with the preset saturation output value (S550).

As a result of the comparison, based on the fact that the resistive output value is greater than the saturation output value (e.g., in the high concentration state), the processor 350 may estimate the final concentration of the hydrogen based on the thermal conduction output value (S560).

As a result of the comparison of operation S550, the processor 350 may determine (e.g., estimate) the concentration of the hydrogen based on the resistive output value, based on the fact that the resistive output value is smaller than or equal to the saturation output value (e.g., in the low concentration state) (S570). The processor 350 may identify an expected thermal conduction output value corresponding to the estimated concentration of the hydrogen and perform humidity correction based on a difference value between the identified expected thermal conduction output value and a currently acquired thermal conduction output value (S575). The processor 350 may compensate for the thermal conduction output value based on the corrected humidity to estimate the final concentration of the hydrogen (S580).

The processor 350 may output the environmental temperature and the estimated final concentration of the hydrogen in the high concentration state and output the environmental temperature, the estimated final concentration of the hydrogen, and the humidity in the low concentration state (S590).

The present disclosure may correct the concentration of the hydrogen and the humidity by combining influences on the concentration of the hydrogen and the humidity in the two manners if an output value of the hydrogen detecting material in a non-saturation area is greater than or equal to a level at which the influence on the humidity may not be excluded. If a current section is an uncorrectable section due to nonsaturation-saturation overlapping, only the concentration of the hydrogen may be estimated while the current section is treated as the saturation section. In this case, the estimation of the humidity and the concentration of the hydrogen according to excessive saturation output determination may be performed using pre-derived simultaneous equations or using pre-constructed mapping data.

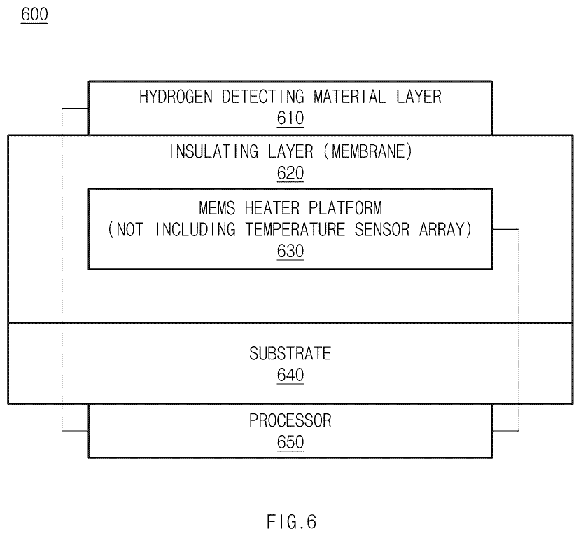

FIG. 6 is a view for describing an example configuration of a hybrid hydrogen sensor.

Referring to FIG. 6, a hybrid hydrogen sensor 600 may include a hydrogen detecting material layer 610, an insulating layer (membrane) 620, a substrate 640, and a processor 650.

A MEMS heater platform 630 may be formed inside the insulating layer 620. The MEMS heater platform 630 may not include a component for sensing a temperature. That is, the MEMS heater platform 630 may be implemented to have only a heater array and no temperature sensors (e.g., a temperature sensor array).

The MEMS heater platform 630 may be disposed inside (or between) the insulating layers 620, thereby securing heating performance of the MEMS heater.

As an example, the insulating layer 620 may be formed of a silicon oxide (SiO₂) layer or a silicon nitride (Si₃N₄) layer.

The heater array may be implemented on the same plane through a patterning technique such as photolithography.

The heater array may be implemented in various micro-patterns such as a meander pattern to ensure excellent temperature increasing performance.

The processor 350 may detect an environmental temperature by measuring a resistance value in a state in which power is not applied to the heater array (e.g., in a state in which the heater pattern is turned off).

A metal material used in the MEMS heater array may be pure platinum (Pt) having high linearity TCR and chemical stability and capable of generating Joule heating, but the present disclosure is not limited thereto.

The insulating layer 620 may be disposed on one surface of the substrate 640, and the processor 650 may be disposed on the other surface thereof, but this is merely an example, and the insulating layer 620 and the processor 650 may be arranged not to overlap each other on the same surface of the substrate 640.

The hydrogen detecting material layer 610 may be implemented as pure palladium in the form of a thin film.

The hydrogen detecting material layer 610 and the MEMS heater platform 630 may be electrically connected to the processor 650 through the substrate 640.

The processor 650 may detect the presence or absence of the hydrogen and the concentration of the hydrogen by monitoring a change in a resistance of the hydrogen detecting material layer 610. That is, the processor 650 may perform a hydrogen detecting material-based resistive hydrogen sensing operation to calculate a resistive output value and estimate the current concentration of the hydrogen based on the calculated resistive output value.

The processor 650 may measure the presence or absence of the hydrogen and the concentration of the hydrogen by monitoring a change in a temperature of the MEMS heater platform 630. That is, the processor 650 may performing a MEMS heater-based thermal conduction hydrogen sensing operation to calculate a thermal conduction output value and estimate the current concentration of the hydrogen based on the calculated thermal conduction output value.

The processor 650 may determine whether the current concentration of the hydrogen is in an output saturation state based on chemical reaction saturation characteristics of a hydrogen detecting material used in the hydrogen detecting material layer 610.

The processor 650 may determine whether to enter a saturation output state based on comparison between a preset saturation output value and the resistive output value according to the corresponding hydrogen detecting material.

The processor 650 may determine that a current state is a saturation output state when the resistive output value is greater than the preset saturation output value, and determine that the current state is a normal output state when the resistive output value is smaller than or equal to the preset saturation output value. In the following description, the saturation output state and the normal output state are used interchangeably as a high concentration state and a low concentration state, respectively.

If the current concentration of the hydrogen is in the saturation output state (e.g., if the current concentration of the hydrogen is in the high concentration state), the processor 650 may estimate the current concentration of the hydrogen based on the thermal conduction output value.

If the current concentration of the hydrogen is in the normal output state (e.g., if the current concentration of the hydrogen is in the low concentration state), the processor 650 may correct the concentration of the hydrogen and humidity based on the thermal conduction output value and the resistive output value.

As an example, the processor 650 may estimate the concentration of the hydrogen based on the resistive output value in the low concentration state and then calculate an expected thermal conduction output value A1 corresponding to the estimated concentration of the hydrogen by applying the expected thermal conduction output value A1 to a preset linear equation as indicated by reference numeral 220 of FIG. 2. The processor 650 may estimate a humidity value based on a difference value between the expected thermal conduction output value A1 and a currently estimated thermal conduction output value A2. Since an influence on humidity is included (e.g., reflected) in the case of the thermal conduction output value, the processor 350 may correct the A2 based on the estimated humidity value and determine the concentration of the hydrogen corresponding to the corrected A2 as a final concentration of the hydrogen.

The processor 650 may finally output information on the environmental temperature, the estimated (or corrected) humidity, and the concentration of the hydrogen. The information output by the processor 650 may be output through a screen of measurement equipment connected to the hybrid hydrogen sensor 600, as shown in FIG. 10, which will be described below.

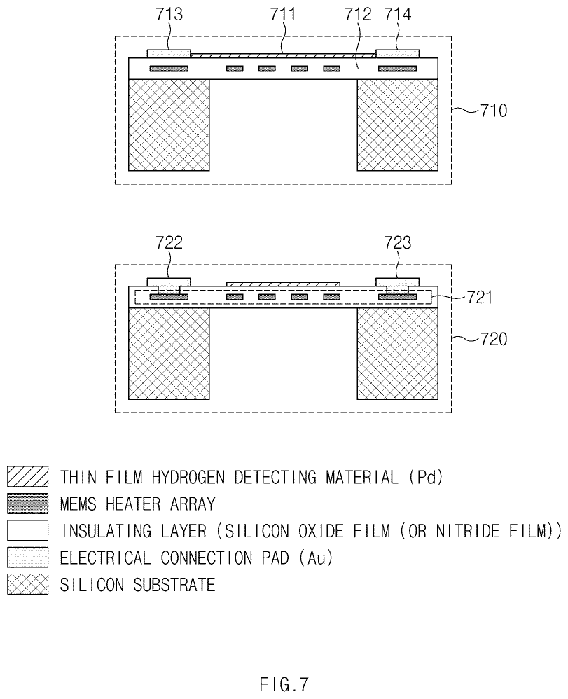

FIG. 7 is a view for describing an example structure of the hybrid hydrogen sensor.

FIG. 7 shows electrical connection cross-sections with the MEMS heater platform of the hybrid hydrogen sensor having only the heater array unlike FIG. 4.

Referring to reference numeral 710, a hydrogen detecting material 711 in the form of a thin film may be disposed on one surface of an insulating layer 712, and both ends of the thin film hydrogen detecting material 711 may be connected to first electrical connection pads 713 and 714 formed on the one surface of the insulating layer 712.

Referring to reference numeral 720, a heater array of a MEMS heater platform 721 may be connected to second electrical connection pads 722 and 723 formed on the one surface of the insulating layer 712. The heater array may be formed at (e.g., in or on) the MEMS heater platform 721.

Palladium may be used as the thin film hydrogen detecting material, and platinum may be used as the heater array.

A silicon oxide film or a silicon nitride film may be used as the insulating layer.

The electrical connection pads may be made of gold (Au), but the present disclosure is not limited thereto, and the electrical connection pads may be made of any one of silver (Ag), copper (Cu), aluminum (Al), and an alloy material using them according to a design of the those skilled in the art and the use and purpose of the hybrid hydrogen sensor.

A silicon substrate may be used as the substrate.

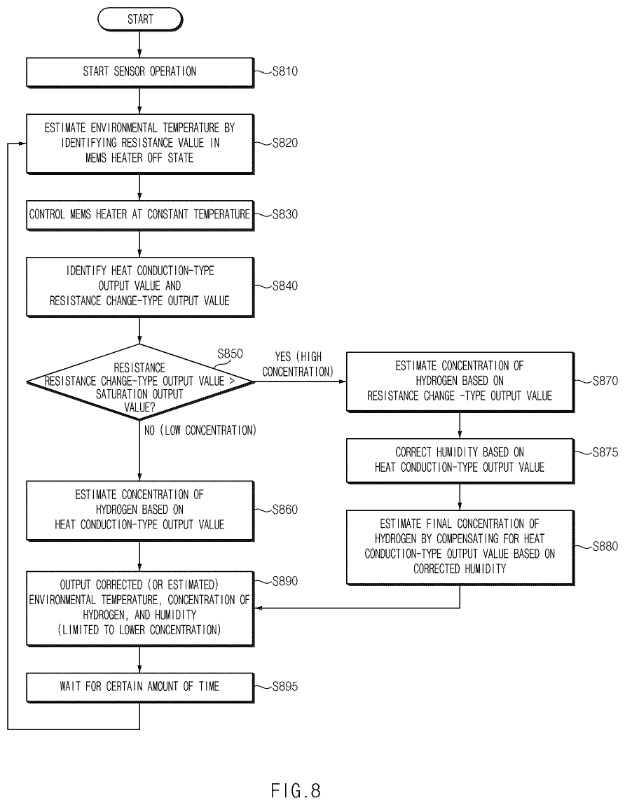

FIG. 8 is a flowchart showing an example method of controlling the hybrid hydrogen sensor.

The controlling method of FIG. 8 may be a method performed by the processor 650 of FIG. 6.

Referring to FIGS. 6 and 8, the processor 650 may start a sensor operation when power is applied to the hybrid hydrogen sensor 600 (S810).

The processor 650 may estimate the environmental temperature based on the resistance value measured in a state in which supply of a current to the MEMS heater is blocked (or a predefined micro-current is suppled)-hereinafter, this state is referred to as a “heater OFF state” (S820). Here, the micro-current refers to a small current that does not affect a temperature increase, which is like a current used for general multi-meter measurement, and is not considered as a heater ON state.

The processor 650 may perform constant temperature control for maintaining the MEMS heater at a predefined normal temperature by performing resistance (temperature) feedback control after a normal current starts to be supplied to a heater array of the MEMS heater platform 630.

The processor 650 may acquire (identify) a MEMS heater-based thermal conduction output value and a hydrogen detecting material-based resistive output value (S840).

The processor 650 may compare the resistive output value with the preset saturation output value (S850).

As a result of the comparison, based on the fact that the resistive output value is greater than the saturation output value (e.g., in the high concentration state), the processor 650 may estimate the final concentration of the hydrogen based on the thermal conduction output value (S860).

As a result of the comparison of operation S850, the processor 650 may estimate the concentration of the hydrogen based on the resistive output value based on the fact that the resistive output value is smaller than or equal to the saturation output value (e.g., in the low concentration state) (S870). Next, the processor 650 may identify an expected thermal conduction output value corresponding to the estimated concentration of the hydrogen and perform humidity correction based on a difference value between the identified expected thermal conduction output value and a currently acquired thermal conduction output value (S875). The processor 650 may compensate for the thermal conduction output value based on the corrected humidity to estimate the final concentration of the hydrogen (S880).

The processor 650 may output the environmental temperature and the estimated final concentration of the hydrogen in the high concentration state and output the environmental temperature, the estimated final concentration of the hydrogen, and the humidity in the low concentration state (S890).

The processor 650 may enter operation S820 after waiting for a certain time (e.g., 1 second) to stabilize temperatures of the MEMS heater and the hydrogen detecting material. As an example, the processor 650 may set a timer for a predetermined time and may enter operation S820 when the timer is expired.

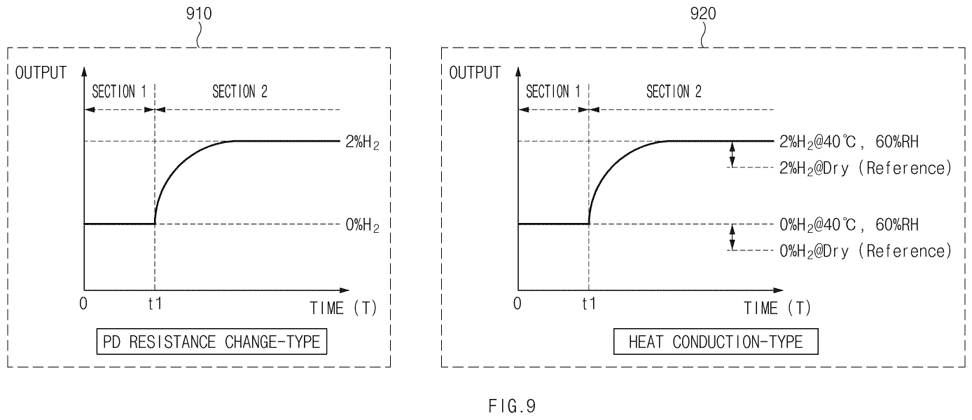

FIG. 9 is an example output of a resistive hydrogen sensor and a thermal conduction hydrogen sensor.

FIG. 9 shows outputs of the resistive hydrogen sensor and the thermal conduction hydrogen sensor when it is assumed that an ambient environment temperature, humidity, and concentration of hydrogen are 40° C., 60% RH, and 0% to 2% H2, respectively.

In addition, a thin film palladium having a saturation concentration value of 2% may be applied as the hydrogen detecting material used in the resistance change type an examples shown in FIG. 9.

When a hydrogen detecting material having a different structure other than the thin film palladium is applied, hydrogen reaction characteristics may vary depending on a type and structure thereof. Here, the hydrogen reaction characteristics may include saturation, a saturation concentration, a humidity effect, and the like, but the present disclosure is not limited thereto.

Reference numeral 910 shows output of the palladium-based resistive hydrogen sensor, and reference numeral 920 shows output of the MEMS heater-based thermal conduction hydrogen sensor.

Referring to reference numeral 910, the palladium resistive hydrogen sensor has an output value corresponding to a hydrogen concentration of 0% (0% H2) during an atmospheric exposure time (section 1) in which there is no hydrogen leakage and has an output value corresponding to a hydrogen concentration of 2% (2% H2) in a hydrogen leakage section (section 2) when hydrogen leakage starts at a time t1.

Referring to reference numeral 920, the thermal conduction hydrogen sensor has an output value corresponding to a hydrogen concentration of 0% (0% H2) in Dry condition assumption during the atmospheric exposure time (section 1) in which there is no hydrogen leakage and has an output value corresponding to a hydrogen concentration of 2% (2% H2) in the Dry condition assumption in the hydrogen leakage section (section 2) when hydrogen leakage starts at the time t1.

Considering the provided environmental conditions, the thermal conduction hydrogen sensor may have output values corresponding to 0% H₂, 0% H₂ that is greater than the output value in the Dry condition assumption by a predetermined level, and a 60% RH condition.

In this case, the output values in the hydrogen detecting method are values measured after the environmental temperature is identified and constant temperature control is completed according to the above-described method.

In this case, after 0% H₂ is identified based on the output value of the palladium resistive hydrogen sensor, a degree of output influence at 60% RH may be identified by erasing the thermal conduction from output value the output value corresponding to 0% H₂ and the Dry condition. A relative humidity (60% RH) may be estimated based on the identified information on the output influence and the identified environmental temperature value.

Thereafter, the concentration of the hydrogen and the humidity may be estimated in the same manner as a 0% H₂ condition during the atmospheric exposure time (section 2) that is changed and maintained under a 2% hydrogen including condition due to occurrence of the hydrogen leakage.

FIG. 10 is an example output of the hybrid hydrogen sensor.

FIG. 10 shows an output example of the hybrid hydrogen sensor under the same environmental condition as FIG. 9.

As shown in FIG. 10, the output of the hybrid hydrogen sensor may include at least one of information on the final concentration of the hydrogen, information on the environmental temperature, and information on the humidity, which are estimated based on information on whether saturation output is exceeded (and/or low/high concentration state information), the resistive output value, and the thermal conduction output value. Here, the humidity may be output only when the concentration of the hydrogen is in the low concentration state.

The term “module” or “unit” used in the specification means a software and/or hardware component, and the “module” or “unit” performs certain operations/functions/roles. However, the “module” or “unit” is not construed as being limited to software or hardware. The “module” or “unit” may be configured to be in an addressable storage medium or to execute one or more processors.

Therefore, as an example, the “module” or “unit” may include at least one of components such as software components, object-oriented software components, class components, and task components, processes, functions, attributes, procedures, sub-routines, segments of program codes, drivers, firmware, micro-codes, circuits, data, databases, data structures, tables, arrays, or variables. Functions provided in the components, “modules”, or “units” may be combined into a smaller number of components, “modules”, or “units” or further divided into additional components, “modules”, or “units”.

In the present disclosure, the “module” or “unit” may be realized as a processor and a memory. The “processor” should be widely construed to include a general-purpose processor, a central processing unit (CPU), a microprocessor, a digital signal processor (DSP), a microcontroller, a state machine, or the like. In some environments, the “processor” may refer to an application-specific integrated circuit (ASIC), a programmable logic device (PLD), or a field-programmable gate array (FPGA), and the like. For example, the “processor” may refer to a combination of processing devices such as a combination of a DSP and a microprocessor, a combination of a plurality of microprocessors, a combination of one or more microprocessors combined with a DSP core, or any other such combination. Moreover, the “memory” should be widely construed to include any electronic component capable of storing electronic information. The “memory” may refer to various types of processor-readable medium such as a random access memory (RAM), a read only memory (ROM), a non-volatile random access memory (NVRAM), a programmable read only memory (PROM), an erasable programmable read only memory (EPROM), an electrically erasable programmable read only memory (EEPROM), a flash memory, a magnetic or optical data storage device, and registers. When the processor can read information from a memory and/or record the information in the memory, the memory may be in a state of electronic communication with a processor. Memory integrated into a processor is in a state of electronic communication with the processor.

The one or more features described herein may be provided as a computer program stored in a computer-readable recording medium in order to be executed on a computer. The medium may either continuously store a computer-executable program or temporarily store the program for execution or download. Furthermore, the medium may be a variety of recording or storage means in the form of a single hardware device or multiple combined hardware devices, and is not limited to media directly connected to some computer system but may also be distributed across a network. Examples of such media include magnetic media such as a hard disk, a floppy disk, or a magnetic tape, optical recording media such as a CD-ROM or a DVD, magneto-optical media such as a floptical disk, and a ROM, RAM, or flash memory, among others, configured to store program instructions. Additional examples of such media include media or storage media that are managed by an app store that distributes applications or by various other sites or servers that provide or distribute software.

In a hardware implementation, processing units used for performing the techniques may be implemented within one or more ASICS, DSPs, digital signal processing devices, programmable logic devices, field-programmable gate arrays, processors, controllers, microcontrollers, microprocessors, electronic devices, or computers or combinations thereof designed to perform the functions described in the present disclosure.

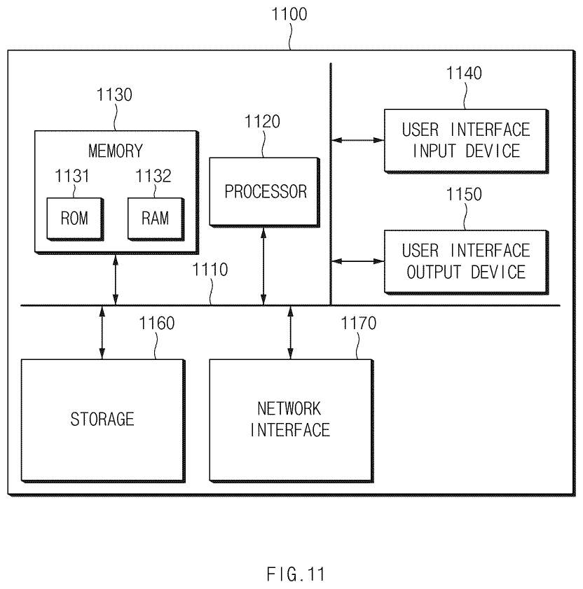

FIG. 11 shows an example computing device or controller.

Referring to FIG. 11, a computing device 1100 (e.g., a computing device or a controller) may include at least one of at least one processor 1120, a memory 1130, a user interface input device 1140, a user interface output device 1150, storage 1160, and a network interface 1170 that are connected through a bus 1110.

The processor 1120 may be a central processing unit (CPU) or a semiconductor device that processes commands stored in the memory 1130 and/or the storage 1160. The memory 1130 and the storage 1160 may include various types of volatile or nonvolatile storage media. For example, the memory 1130 may include a read only memory (ROM) 1131 and a random access memory (RAM) 1132.

Thus, the operations of the method (or procedure) or the algorithm described in connection with one or more embodiments disclosed herein may be directly implemented by hardware modules, software modules, or a combination of both the hardware modules and the software modules, which are executed by the processor 1120. The software module may reside in a storage medium (that is, the memory 1130 and/or the storage 1160) such as a RAM, a flash memory, a ROM, an erasable programmable ROM (EPROM), an electrically erasable programmable ROM (EEPROM), a register, a hard disk, a removable disk, and a compact disc ROM (CD-ROM). As an example, the processor 1120 may constitute a portion of the above-described fuel cell vehicle system.

An exemplary storage medium may be coupled to the processor 1120, and the processor 1120 may read information from the storage medium and write information in the storage medium. In another manner, the storage medium may be formed integrally with the processor 1120. The processor and the storage medium may be implemented in the form of a main control unit (MCU). The MCU may be disposed separately from a sensor detecting element on a PCB substrate in a hybrid hydrogen sensor product. In particular, when temperature measurement and temperature increase are implemented through the heater array, a separate circuit component for variable driving control, which is separate from the MCU, may be included.

According to an aspect of the present disclosure, a method of controlling a hybrid hydrogen sensor includes driving a first sensor and a second sensor when power is applied, obtaining a resistance change-type output value and a thermal conduction-type output value from the first sensor and the second sensor, respectively, comparing the resistance change-type output value with a predetermined saturation output value, and estimating at least one of a concentration of hydrogen and humidity based on at least one of the thermal conduction-type output value and the resistance change-type output value according to the comparison result.

The first sensor, which is a hydrogen detecting material-based resistance change-type hydrogen sensor, and the second sensor, which is a micro heater platform-based thermal conduction-type hydrogen sensor, may be implemented as a single element.

When the resistance change-type output value is greater than the saturation output value as the comparison result, the concentration of the hydrogen may be estimated based on the thermal conduction-type output value.

When the resistance change-type output value is smaller than or equal to the saturation output value as the comparison result, the humidity may be estimated based on the resistance change-type output value and the thermal conduction-type output value, and the concentration of the hydrogen may be estimated based on the estimated humidity.

The hybrid hydrogen sensor may include a micro heater platform, the method may further include detecting an environmental temperature based on a resistance value of a temperature sensor pattern formed in the micro heater platform, and the humidity may be estimated further based on the environmental temperature.

The method may further include performing constant temperature control by applying a current to a heater pattern formed on the micro heater platform after detecting the environmental temperature, and the resistance change-type output value and the thermal conduction-type output value may be acquired based on the completed constant temperature control.

The method may further include outputting an environmental temperature, the humidity, and the concentration of the hydrogen based on a low concentration state in which the resistance change-type output value is smaller than or equal to the saturation output value and outputting the environmental temperature and the concentration of the hydrogen based on a high concentration state in which the resistance change-type output value is greater than the saturation output value. The method may further include driving a predetermined timer after the outputting, wherein the method may re-enter detecting the environmental temperature based on the expired timer.

The hybrid hydrogen sensor may include a hydrogen detecting material and a micro heater platform, and when the micro heater platform does not include a temperature sensor pattern, an environmental temperature may be estimated by measuring a resistance value of a heater pattern formed on the micro heater platform in a heater OFF state.

The first sensor may include a hydrogen detecting material, and the saturation output value may be determined based on hydrogen reaction characteristics of the hydrogen detecting material.

According to another aspect of the present disclosure, a hybrid hydrogen sensor includes an insulating layer in which a micro heater platform is formed, a substrate disposed on one surface of the insulating layer, a hydrogen detecting material layer disposed on the other surface of the insulating layer, and a processor disposed on one side of the substrate, wherein the processor compares a hydrogen detecting material layer-based resistance change-type output value with a predetermined saturation output value and estimates at least one of a concentration of hydrogen and humidity based on at least one of the resistance change-type output value and a micro heater platform-based thermal conduction-type output value according to the comparison result.

The micro heater platform may include a heater pattern and a temperature sensor pattern formed on the same plane.

When the resistance change-type output value is greater than the saturation output value as the comparison result, the processor may estimate the concentration of the hydrogen based on the thermal conduction-type output value.

When the resistance change-type output value is smaller than or equal to the saturation output value as the comparison result, the processor may estimate the humidity based on the resistance change-type output value and the thermal conduction-type output value and estimate the concentration of the hydrogen based on the estimated humidity.

The processor may detect an environmental temperature based on a resistance value of the temperature sensor pattern and estimate the humidity further based on the environmental temperature.

The processor may perform constant temperature control by applying a current to the heater pattern after detecting the environmental temperature and acquire the resistance change-type output value and the thermal conduction-type output value based on the completed constant temperature control.

The processor may output the environmental temperature and the concentration of the hydrogen based on a high concentration state in which the resistance change-type output value is greater than the saturation output value and output the environmental temperature, the concentration of the hydrogen, and the humidity based on a low concentration state in which the resistance change-type output value is smaller than or equal to the saturation output value.

The processor may drive a predetermined timer after the outputting and control the process to be performed again from the detecting of the environmental temperature based on the expired timer.

When the micro heater platform does not include a temperature sensor pattern, the processor may estimate an environmental temperature by measuring a resistance value of a heater pattern formed on the micro heater platform in a heater OFF state.

The saturation output value may be determined based on hydrogen n reaction characteristics of a hydrogen detecting material forming the hydrogen detecting material layer, and the hydrogen detecting material may include palladium.

The present technology provides a hybrid hydrogen sensor having measurement accuracy and a wide detection range for a concentration of hydrogen, and a method of controlling the same.

Further, the present technology provides a hybrid hydrogen sensor capable of measuring a wide range of a concentration of hydrogen in a hybrid detecting manner by coupling a hydrogen detecting material-based resistive hydrogen sensor and a MEMS heater-based thermal conduction-type hydrogen sensor into a single element, and a method of controlling the same.

Further, the present technology provides a hydrogen sensor having high accuracy and a wide detecting range without an additional compensation element by applying a hybrid detecting method on a single element.

Further, the present technology may determine whether a sensor is normal based on comparison between output values of two sensing logics on a single element.

Further, the present technology provides a hydrogen sensor having excellent price competitiveness.

In addition, various effects directly or indirectly identified though the present document may be provided.

The above description is merely illustrative of the technical spirit of the present disclosure, and those skilled in the art to which the present disclosure belongs may make various modifications and changes without departing from the essential features of the present disclosure.

Thus, one or more embodiments disclosed in the present disclosure are not intended to limit the technology spirit of the present disclosure, but are intended to describe the present disclosure, and the scope of the technical spirit of the present disclosure is not limited by these example embodiments. The scope of protection of the present disclosure should be interpreted by the appended claims, and all technical spirits within the scope equivalent thereto should be interpreted as being included in the scope of the present disclosure.

What is claimed is:

1. A method comprising:
applying power to a hybrid hydrogen sensor comprising a resistive hydrogen sensor and a thermal conduction hydrogen sensor;
obtaining, from the resistive hydrogen sensor, a first output value;
obtaining, from the thermal conduction hydrogen sensor, a second output value;
comparing the first output value with a predetermined saturation output value;
estimating at least one of a concentration of hydrogen or humidity based on one or more selections, according to a result of the comparison, from the first output value and the second output value; and
outputting, based on the estimating, a signal indicating the at least one of the concentration of hydrogen or the humidity for a fuel cell.

2. The method of claim 1, wherein the resistive hydrogen sensor is based on a hydrogen detecting material, wherein the thermal conduction hydrogen sensor is based on a micro heater platform, and wherein the resistive hydrogen sensor and the thermal conduction hydrogen sensor are implemented as a single element.

3. The method of claim 1, wherein the estimating of the at least one of the concentration of hydrogen or the humidity comprises:
estimating, based on the result of the comparison indicating that the first output value is greater than the predetermined saturation output value, the concentration of hydrogen based on the second output value.

4. The method of claim 1, wherein the estimating of the at least one of the concentration of hydrogen or the humidity comprises:
based on the result of the comparison indicating that the first output value is less than or equal to the predetermined saturation output value:
estimating the humidity based on the first output value and the second output value; and
estimating the concentration of hydrogen based on the estimated humidity.

5. The method of claim 4, wherein the hybrid hydrogen sensor comprises a micro heater platform,
wherein the method further comprises determining an environmental temperature based on a resistance value of a temperature sensor array formed at the micro heater platform, and
wherein the humidity is estimated further based on the environmental temperature.

6. The method of claim 5, further comprising:
performing constant temperature control by applying a current to a heater array formed at the micro heater platform after the determining of the environmental temperature,
wherein the first output value and the second output value are obtained based on the performing of the constant temperature control.

7. The method of claim 1, further comprising one of:
outputting, based on a low concentration state in which the first output value is less than or equal to the predetermined saturation output value, an environmental temperature, the humidity, and the concentration of hydrogen; or
outputting, based on a high concentration state in which the first output value is greater than the predetermined saturation output value, the environmental temperature and the concentration of hydrogen.

8. The method of claim 7, further comprising:
setting a timer after the outputting; and
determining, based on the timer being expired, a second environmental temperature.

9. The method of claim 1, wherein the hybrid hydrogen sensor comprises a hydrogen detecting material and a micro heater platform, and wherein the method further comprises:
estimating, based on a determination that the micro heater platform does not include a temperature sensor array, an environmental temperature by measuring a resistance value of a heater array formed at the micro heater platform in a heater OFF state.

10. The method of claim 1, wherein the resistive hydrogen sensor comprises a hydrogen detecting material, and wherein the predetermined saturation output value is determined based on hydrogen reaction characteristics of the hydrogen detecting material.

11. A hybrid hydrogen sensor comprising:
an insulating layer in which a micro heater platform is formed;
a substrate disposed on a first surface of the insulating layer;
a hydrogen detecting material layer disposed on a second surface of the insulating layer;
a processor disposed on the substrate; and
a memory storing at least one instruction that, when executed by the processor communicating with the memory, is configured to cause the hybrid hydrogen sensor:
compare a first sensor output value that is based on the hydrogen detecting material layer with a predetermined saturation output value;
estimate at least one of a concentration of hydrogen or humidity based on one or more selections, according to a result of the comparison, from the first sensor output value and a second sensor output value that is based on the micro heater platform; and
output, based on the estimation, a signal indicating the at least one of the concentration of hydrogen or the humidity for a fuel cell.

12. The hybrid hydrogen sensor of claim 11, wherein the micro heater platform comprises a heater array and a temperature sensor array formed on a same plane.

13. The hybrid hydrogen sensor of claim 12, wherein, the at least one instruction, when executed by the processor communicating with the memory, is configured to cause the hybrid hydrogen sensor to estimate the at least one of the concentration of hydrogen or the humidity by:
estimating, based on the result of the comparison indicating that the first sensor output value is greater than the predetermined saturation output value, the concentration of hydrogen based on the second sensor output value.

14. The hybrid hydrogen sensor of claim 13, wherein the at least one instruction, when executed by the processor communicating with the memory, is configured to cause the hybrid hydrogen sensor to estimate the at least one of the concentration of hydrogen or the humidity by:
based on the result of the comparison indicating that the first sensor output value is less than or equal to the predetermined saturation output value:
estimating the humidity based on the first sensor output value and the second sensor output value; and
estimating the concentration of hydrogen based on the estimated humidity.

15. The hybrid hydrogen sensor of claim 14, wherein the at least one instruction, when executed by the processor communicating with the memory, is configured to further cause the hybrid hydrogen sensor to:
determining an environmental temperature based on a resistance value of the temperature sensor array, and
wherein the estimating of the humidity is further based on the environmental temperature.

16. The hybrid hydrogen sensor of claim 15, wherein the at least one instruction, when executed by the processor communicating with the memory, is configured to further cause the hybrid hydrogen sensor to:
perform constant temperature control by applying a current to the heater array after the determining of the environmental temperature; and
obtain the first sensor output value and the second sensor output value based on the performing of the constant temperature control.

17. The hybrid hydrogen sensor of claim 11, wherein the at least one instruction, when executed by the processor communicating with the memory, is configured to further cause the hybrid hydrogen sensor to perform one of:
outputting, based on a high concentration state in which the first sensor output value is greater than the predetermined saturation output value, an environmental temperature and the concentration of hydrogen; or
outputting, based on a low concentration state in which the first sensor output value is less than or equal to the predetermined saturation output value, the environmental temperature, the concentration of hydrogen, and the humidity.

18. The hybrid hydrogen sensor of claim 17, wherein the at least one instruction, when executed by the processor communicating with the memory, is configured to further cause the hybrid hydrogen sensor to:
set a timer after the outputting; and
determine, based on the timer being expired, a second environmental temperature.

19. The hybrid hydrogen sensor of claim 11, wherein the at least one instruction, when executed by the processor communicating with the memory, is configured to further cause the hybrid hydrogen sensor to:
estimate, based on a determination that the micro heater platform does not include a temperature sensor array, an environmental temperature by measuring a resistance value of a heater array formed at the micro heater platform in a heater OFF state.

20. A hybrid hydrogen sensor comprising:
a micro heater platform;
a hydrogen detecting material layer;
a processor; and
a memory storing at least one instruction that, when executed by the processor communicating with the memory, is configured to cause the hybrid hydrogen sensor to:
compare a first sensor output value that is based on the hydrogen detecting material layer with a predetermined saturation output value;
estimate at least one of a concentration of hydrogen or humidity based on one or more selections, according to a result of the comparison, from the first sensor output value and a second sensor output value that is based on the micro heater platform; and
output, based on the estimation, a signal indicating the at least one of the concentration of hydrogen or the humidity for a fuel cell.
